(19) United States
(12) Patent Application Publication
(10) Pub. No.: US 2013/0156768 A1
(43) Pub. Date: Jun. 20, 2013

(54) COMBINATION OF ANTI-CTLA4 ANTIBODY WITH BRAF INHIBITORS FOR THE SYNERGISTIC TREATMENT OF PROLIFERATIVE DISEASES

(71) Applicants: Maria Jure-Kunkel, Plainsboro, NJ (US); Jon Marc Wigginton, Collegeville, PA (US); Ashok Kumar Gupta, Plainsboro, NJ (US)

(72) Inventors: Maria Jure-Kunkel, Plainsboro, NJ (US); Jon Marc Wigginton, Collegeville, PA (US); Ashok Kumar Gupta, Plainsboro, NJ (US)

(73) Assignee: Bristol-Myers Squibb Company, Princeton, NJ (US)

(21) Appl. No.: 13/818,714

(22) PCT Filed: Aug. 25, 2011
(86) PCT No.: PCT/US11/49075

§ 371 Date: Feb. 25, 2013

Related U.S. Application Data
(60) Provisional application No. 61/377,297, filed on Aug. 26, 2010.
(60) Provisional application No. 61/379,152, filed on Sep. 1, 2010.

(51) Int. Cl. A61K 39/395; A61K 31/437; A61K 31/4184

(52) U.S. Cl. CPC A61K 39/3955; A61K 31/4184; A61K 31/437
USPC 424/135.1; 424/142.1; 424/144.1; 424/173.1; 514/44 A; 514/19.3; 514/1.1

(57) ABSTRACT

Compositions and methods are disclosed which are useful of the treatment and prevention of proliferative disorders.

This application claims benefit to provisional application U.S. Ser. No. 61/377,297 filed Aug. 26, 2010; and to provisional application U.S. Ser. No. 61/379,152, filed Sep. 1, 2010; under 35 U.S.C. §119(e). The entire teachings of the referenced applications are incorporated herein by reference.

### BRIEF DESCRIPTION OF THE SEQUENCE LISTING

Incorporated herein by reference in its entirety is a Sequence Listing entitled, “11638PCT_ST25.txt”, comprising SEQ ID NO:1 through SEQ ID NO:4, which include nucleic acid and/or amino acid sequences disclosed herein. The Sequence Listing has been submitted herewith in IBM/PC MS-DOS text format via EFS, was first created on Aug. 22, 2011, and is 4 KB in size.

### FIELD OF THE INVENTION

This invention relates to the fields of oncology and improved therapy regimens.

### BACKGROUND OF THE INVENTION

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action.

One type of chemotherapeutic is referred to as a metal coordination complex. It is believed this type of chemotherapeutic forms predominantly inter-strand DNA cross links in the nuclei of cells, thereby preventing cellular replication. As a result, tumor growth is initially repressed, and then reversed. Another type of chemotherapeutic is referred to as an alkylating agent. These compounds function by inserting foreign compositions or molecules into the DNA of dividing cancer cells. As a result of these foreign moieties, the normal functions of cancer cells are disrupted and proliferation is prevented. Another type of chemotherapeutic is an antineoplastic agent. This type of agent prevents, kills, or blocks the growth and spread of cancer cells. Still other types of anticancer agents include nonsteroidal aromastase inhibitors, bifunctional alkylating agents, etc.

Chemoimmunotherapy, the combination of chemotherapeutic and immunotherapeutic agents, is a novel approach for the treatment of cancer which combines the effects of agents that directly attack tumor cells producing tumor cell necrosis or apoptosis, and agents that modulate host immune responses to the tumor. Chemotherapeutic agents could enhance the effect of immunotherapy by generating tumor antigens to be presented by antigen-presenting cells creating a “polyvalent” tumor cell vaccine, and by distorting the tumor architecture, thus facilitating the penetration of the immunotherapeutic agents as well as the expanded immune population.

Ipilimumab is a human anti-human CTLA-4 antibody which blocks the binding of CTLA-4 to CD80 and CD86 expressed on antigen presenting cells and thereby, blocking the negative downregulation of the immune responses elicited by the interaction of these molecules. Since ipilimumab does not recognize mouse CTLA-4, an anti-mouse CTLA-4 antibody (clone UC10-4F10) was used in the studies presented here to investigate the effect of CTLA-4 blockade with chemotherapeutic agents.

The Ras-Raf-MEK-ERK signaling pathway has been implicated in human oncogenesis (Halilovic et al., Curr. Opin. Pharmacol., 8:419-426 (2008); McCubrey et al., Curr. Opin. Investig. Drugs, 9:614-630 (2008); and Michaloglou et al., Oncogene, 27:877-895 (2008)). This pathway normally connects extracellular signals, such as growth factors and hormones, to the nucleus, leading to the expression of genes that regulate cell proliferation, differentiation, and survival (McCubrey et al.). When a ligand binds to its receptor tyrosine kinase on the plasma membrane, it stimulates the activity of Ras. One major effector of Ras is the Raf family of serine/threonine kinases, which comprises A-Raf, BRAF, and C-Raf (Beck et al., Nucleic Acids Res., 15:595-609 (1987); Bonner et al., Mol. Cell. Biol., 5:1400-1407 (1985); Huebner et al., Proc. Natl. Acad. Sci. USA, 83:3934-3938 (1986); and Ikawa et al., Mol. Cell. Biol., 8:2651-2654 (1988)). Raf proteins signal through phosphorylation and activation of a downstream kinase, mitogen-activated protein (MAP)/extracellular signal-regulated kinase (ERK) kinase (MEK), which subsequently phosphorylates and activates ERK (Macdonald et al., Mol. Cell. Biol., 13:6615-6620 (1993)). The Ras-Raf-MEK-ERK pathway may be constitutively activated in human cancers through mutations in Ras or Raf (Halilovic et al., McCubrey et al.; and Michaloglou et al.). Based on its association with human cancers, BRAF has been a target for therapeutic treatment of cancer.

In the studies described herein, the combination of a BRAF inhibitor with a CTLA-4 inhibitor was investigated in several tumor models.

The present inventors have discovered for the first time the synergistic benefit of combining a BRAF inhibitor with an anti-CTLA-4 inhibitor for the treatment of proliferative diseases, and in particular, the synergistic benefit of sequentially administering, and in some cases concurrently administering, a BRAF inhibitor with an anti-CTLA-4 inhibitor for the treatment of proliferative diseases. It is an object of the invention to provide efficacious combination chemotherapeutic treatment regimens wherein one or more BRAF inhibitors is combined with one or more anti-CTLA4 agents for the treatment of proliferative diseases.

### SUMMARY OF THE INVENTION

The present invention provides a synergistic method for the treatment of anti-proliferative diseases, including cancer, which comprises administering to a mammalian species in need thereof a synergistic, therapeutically effective amount of: (1) Methyl {5-[2-chloro-2-methylphenyl)-1-hydroxy-3-oxo-2,3-dihydro-1H-isoindol-1-yl]-1H-benzimidazol-2-yl}carbamate; and (2) ipilimumab or tremelimumab.

The present invention provides a synergistic method for the treatment of anti-proliferative diseases, including cancer, which comprises administering to a mammalian species in need thereof a synergistic, therapeutically effective amount of: (1) Methyl {5-[2-chloro-2-methylphenyl)-1-hydroxy-3-oxo-2,3-dihydro-1H-isoindol-1-yl]-1H-benzimidazol-2-yl}carbamate; and (2) a co-stimulatory pathway modulator, such as an anti-CTLA4 antagonist.

The present invention provides a synergistic method for the treatment of anti-proliferative diseases, including cancer, which comprises administering to a mammalian species in need thereof a synergistic, therapeutically effective amount of: (1) a BRAF inhibitor; and (2) a co-stimulatory pathway modulator, such as an anti-CTLA4 antagonist.

The present invention provides a synergistic method for the treatment of anti-proliferative diseases, including cancer, which comprises the sequential administration of a synergistic, therapeutically effective amount of: (1) a BRAF inhibitor; and (2) a co-stimulatory pathway modulator, such as an anti-CTLA4 antagonist; to a mammalian species in need thereof, wherein the BRAF inhibitor is administered first, followed by a CTLA-4 antagonist.

The present invention provides a synergistic method for the treatment of anti-proliferative diseases, including cancer, which comprises the sequential administration of a synergistic, therapeutically effective amount of: (1) a BRAF inhibitor; and (2) a co-stimulatory pathway modulator, such as an anti-CTLA4 antagonist; to a mammalian species in need thereof, wherein the BRAF inhibitor is administered first, followed by a CTLA-4 antagonist either alone or in combination with said BRAF inhibitor.

The present invention provides a synergistic method for treating a mammal with cancer comprising the sequential administration of (i) one or more cycles of a chemotherapeutic agent, followed by (ii) one or more cycles of a combination comprising an immunomodulatory agent with said chemotherapeutic agent. In one aspect of the present invention, the immunomodulatory agent is a modulator of the co-stimulatory pathway, and in particular, a CTLA4 antagonist. In another aspect of the present invention, the chemotherapeutic agent is a BRAF inhibitor. In one aspect of the present invention, the immunomodulatory agent is a modulator of the co-stimulatory pathway, and is selected from the group consisting of: Ipilimumab; ORENCIA®; NULOJIX®; CD28 antagonists, CD80 antagonists, CD86 antagonists, and CTLA-4 antagonists.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes. In one aspect of the present invention, the immunomodulatory agent is a modulator of the co-stimulatory pathway, and in particular, a CTLA4 antagonist. In another aspect of the present invention, the chemotherapeutic agent is a BRAF inhibitor. In one aspect of the present invention, the immunomodulatory agent is a modulator of the co-stimulatory pathway, and is selected from the group consisting of: Ipilimumab; ORENCIA®; NULOJIX®; CD28 antagonists, CD80 antagonists, CD86 antagonists, and CTLA-4 antagonists.

The present invention provides a method for treating a patient with cancer with a sequential administration of (i) one or more cycles of a chemotherapeutic agent, followed by (ii) one or more cycles of a combination comprising an immunomodulatory agent with said chemotherapeutic agent, wherein said method optionally comprises an Intervening Period in-between (i) and (ii), wherein said Intervening Period is between 0 days to 24 weeks in time. In one aspect of the present invention, the Intervening Period is between 2 to 8 weeks. In one aspect of the present invention, the Intervening Period is between 3 to 6 weeks. In another aspect of the present invention, the chemotherapeutic agent is a BRAF inhibitor. In one aspect of the present invention, the immunomodulatory agent is a modulator of the co-stimulatory pathway, and is selected from the group consisting of: Ipilimumab; ORENCIA®; NULOJIX®; CD28 antagonists, CD80 antagonists, CD86 antagonists, and CTLA-4 antagonists.

In one aspect, the proliferative disease is one or more cancerous solid tumors such as melanoma, lung cancer, pancreatic cancer, colon cancer, and/or prostate cancer. In another aspect, the proliferative disease is one or more refractory tumors. In yet another aspect, the CTLA-4 antibody is ipilimumab or tremelimumab. In another aspect, the BRAF inhibitor is Methyl {5-[2-chloro-2-methylphenyl)-1-hydroxy-3-oxo-2,3-dihydro-1H-isoindol-1-yl]-1H-benzimidazol-2-yl}carbamate; a V600E BRAF inhibitor; or PLX-4032 (also referred to as VEMURAFENIB™, and marketed by Roche and Plexxikon).

Suitable anti-CTLA4 antagonist agents for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncol., 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

Each of these references is specifically incorporated herein by reference for purposes of describing CTLA-4 antibodies. A preferred clinical CTLA-4 antibody is human monoclonal antibody 10D1 (also referred to as MDX-010 and ipilimumab and available from Medarex, Inc., Bloomsbury, N.J.) is disclosed in WO 01/14424, and is marketed as YERVOY™ by Bristol-Myers Squibb Company.

### BRIEF DESCRIPTION OF THE DRAWINGS

FIG. 1 illustrates results showing antitumor activity of Compound Ia in combination with CTLA-4 mAb when administered concurrently. Compound Ia (100 mg/kg, p.o.) was dosed on days 4, 6, 8, 10, 12, 14 and 16 while CTLA-4 mAb (20 mg/kg, p.o.) was administered on days 5, 9 and 13.

FIG. 2 illustrates results showing antitumor activity of Compound Ia in combination with CTLA-4 mAb following a sequential schedule. Compound Ia (100 mg/kg, p.o.) was dosed on days 4, 6, 8, and 10, while CTLA-4 mAb (20 mg/kg, p.o.) was administered on days 11, 15, and 19.

FIG. 3 illustrates results showing effect of Compound Ia and erlotininb (TARCEVA®) alone or in combination with CTLA-4 mAb in a model of antigen-specific T cell expansion.

FIG. 4 illustrates results showing the antitumor activity of CTLA-4 mAb and Compound Ia, alone or in combination, in the SA1N fibrosarcoma tumor model.

FIG. 5 illustrates results showing the effect of CTLA-4 mAb+/−raf inhibitors or EVRI on OVA-specific T cell responses.

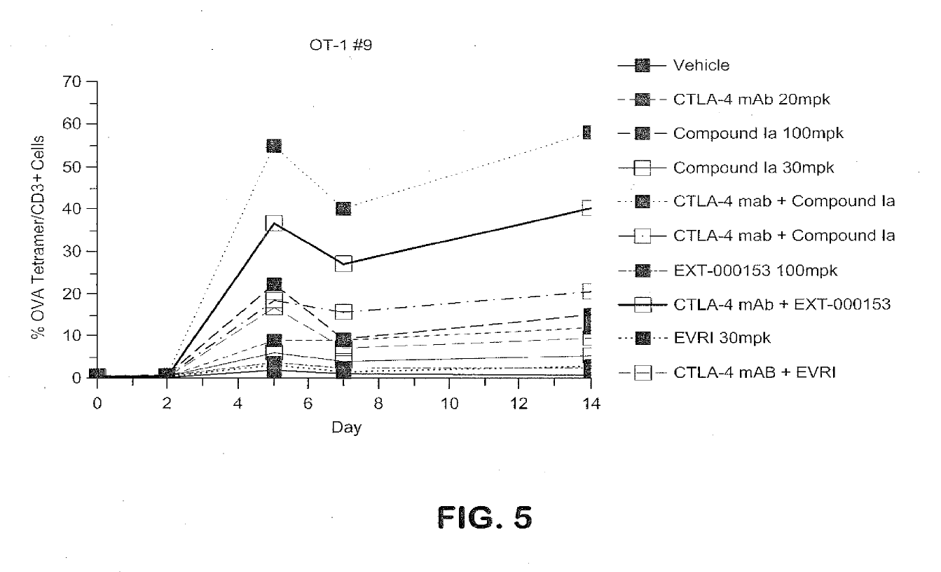

### DETAILED DESCRIPTION OF THE INVENTION

The present invention provides a synergistic method for the treatment of proliferative diseases, including cancer, which comprises administering to a mammalian species in need thereof a synergistic, therapeutically effective amount of: a BRAF inhibitor, and a co-stimulatory pathway modulator, such as an anti-CTLA4 antagonist.

The present invention provides a synergistic, pharmaceutical composition, for the treatment of proliferative diseases, including cancer, which comprises a therapeutically effective amount of: a BRAF inhibitor, and a co-stimulatory pathway modulator, such as an anti-CTLA4 antagonist. In one embodiment, the anti-CTLA4 antagonist is Ipilimumab, and the BRAF inhibitor is Compound Ia.

Optimal T cell activation requires interaction between the T cell receptor and specific antigen (Bretscher, P. et al., Science, 169:1042-1049 (1970)) (the first signal) and engagement of costimulatory receptors on the surface of the T cell with costimulatory ligands expressed by the antigen-presenting cell (APC) (the second signal). Failure of the T cell to receive a second signal can lead to clonal anergy (Schwartz, R. H., Science, 248:1349-1356 (1990)). Two important T cell costimulatory receptors are CD28 and cytotoxic T lymphocyte-associated antigen 4 (CTLA-4, CD152) whose ligands on APC are B7-1 and B7-2 (Linsley, P. S. et al., J. Exp. Med., 173:721-730 (1991); Linsley, P. S. et al., J. Exp. Med., 174:561-569 (1991)). Although CD28 and CTLA-4 are closely related members of the Ig superfamily (Brunet, J. F. et al., Nature, 328:267-270 (1987)), they function antagonistically. CD28 is constitutively expressed on the surface of T cells (Gross, J. A. et al., J. Immunol., 149:380-388 (1992)), and upon engagement with B7-1 or B7-2, enhances the T cell receptor-peptide-MHC signal to promote T cell activation, proliferation, and IL-2 production (Linsley, P. S. et al., J. Exp. Med., 173:721-730 (1991); Alegre, M. L. et al., Nat. Rev. Immunol., 1(3):220-228 (December 2001)). CTLA-4 is not found on resting T cells but is up-regulated for 2-3 days after T cell activation (Lindsten, T. et al., J. Immunol., 151:3489-3499 (1993); Walunas, T. L. et al., Immunity, 1:405-413 (1994)). CTLA-4 also binds to B7-1 and B7-2 but with greater affinity than CD28 (Linsley, P. S. et al., Immunity, 1:793-801 (1994)) and antagonizes T cell activation, interferes with IL-2 production and IL-2 receptor expression, and interrupts cell cycle progression of activated T cells (Walunas, T. L. et al., J. Exp. Med., 183:2541-2550 (1996); Krummel, M. F. et al., J. Exp. Med., 183:2533-2540 (1996); Brunner, M. C. et al., J. Immunol., 162:5813-5820 (1999); Greenwald, R. J. et al., Eur. J. Immunol., 32:366-373 (2002)). The overall T cell response is determined by the integration of all signals, stimulatory and inhibitory.

Because CTLA-4 appears to undermine T cell activation, attempts have been made to block CTLA-4 activity in murine models of cancer immunotherapy. In mice implanted with immunogenic tumors, administration of anti-CTLA-4 Ab enhanced tumor rejection (Leach, D. R. et al., Science, 271:1734-1736 (1996)), although little effect was seen with poorly immunogenic tumors such as SM1 mammary carcinoma or B16 melanoma. Enhanced antitumor immunity was seen when anti-CTLA-4 Ab was given with granulocyte-macrophage colony-stimulating factor (GM-CSF)-transduced B16 cell vaccine and was associated with depigmentation, suggesting that at least part of the antitumor response was antigen-specific against “self” melanocyte differentiation antigens (van Elsas, A. et al., J. Exp. Med., 190:355-366 (1999); van Elsas, A. et al., J. Exp. Med., 194:481-489 (2001)). In a transgenic murine model of primary prostate cancer, administrating anti-CTLA-4 Ab plus GM-CSF-expressing prostate cancer cells reduced the incidence and histological severity of prostate cancer and led to prostatitis in normal mice, again suggesting an antigen-specific immune response against self-antigens in tumor rejection (Hurwitz, A. A. et al., Cancer Res., 60:2444-2448 (2000)). Furthermore, because many human tumor antigens are normal self-antigens, breaking tolerance against self may be critical to the success of cancer immunotherapy. The favorable tumor responses from CTLA-4 blockade in conjunction with tumor vaccines in murine models led to interest in using CTLA-4 blockade in human cancer immunotherapy.

Chemoimmunotherapy, the combination of chemotherapeutic and immunotherapeutic agents, is a novel approach for the treatment of cancer which combines the effects of agents that directly attack tumor cells producing tumor cell necrosis or apoptosis, and agents that modulate host immune responses to the tumor. Chemotherapeutic agents could enhance the effect of immunotherapy by generating tumor antigens to be presented by antigen-presenting cells creating a “polyvalent” tumor cell vaccine, and by distorting the tumor architecture, thus facilitating the penetration of the immunotherapeutic agents as well as the expanded immune population.

Thus, the present invention provides methods for the administration of a BRAF inhibitor in synergistic combination with at least one anti-CTLA4 agent for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof. Preferably, such methods of treating cancer with the treatment regimens of the present invention will result in a diminished incidence of anti-CTLA agent-induced colitis.

The combination of a BRAF inhibitor with at least one co-stimulatory pathway modulator, preferably an anti-CTLA4 agent, may also include the addition of an anti-proliferative cytotoxic agent. Classes of compounds that may be used as anti-proliferative cytotoxic agents include the following:

Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN®), Ifosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

For the purposes of the present invention, a co-stimulatory pathway modulator encompasses one or more of the following: an anti-CTLA4 agent, an anti-CTLA-4 antibody, ipilimumab, and tremelimumab.

For the purposes of the present invention, a BRAF inhibitor may include one or more of the following: Methyl {5-[2-chloro-2-methylphenyl)-1-hydroxy-3-oxo-2,3-dihydro-1H-isoindol-1-yl]-1H-benzimidazol-2-yl}carbamate; a V600E BRAF inhibitor; EXT-000153; RAF265; AZ628; GSK2118436; GSK-1120212; ARQ-197; ARQ-736; NMS-P186; NMS-P349; NMS-P383; NMS-P396; NMS-P730; AB024; E6201; PD-0325901; pyridoimidazolones; RDEA 119; RO-4987655; PLX4032; PLX-3603; selumetinib; TAK-733; and GDC-0879.

Other co-stimulatory pathway modulators of the present invention that may be used in combination with a protein tyrosine kinase inhibitor, either alone or in further combination with other co-stimulatory pathway modulators disclosed herein, or in combination with other compounds disclosed herein include, but are not limited to, the following: agatolimod, NULOJIX®, blinatumomab, CD40 ligand, anti-B7-1 antibody, anti-B7-2 antibody, anti-B7-H4 antibody, AG4263, eritoran, anti-OX40 antibody, ISF-154, and SGN-70; B7-1, B7-2, ICAM-1, ICAM-2, ICAM-3, CD48, LFA-3, CD30 ligand, CD40 ligand, heat stable antigen, B7h, OX40 ligand, LIGHT, CD70 and CD24.

A “immunomodulatory agent” of the present invention generally refers to an agent that either increases or decreases the function of the immune system, and/or as defined elsewhere herein, and includes co-stimulatory pathway modulators, Ipilimumab; ORENCIA®; NULOJIX®; CD28 antagonists, CD80 antagonists, CD86 antagonists, PD1, PDL1, CD137, 41BB, and CTLA-4 antagonists, among others disclosed herein, and may include, for example, a co-stimulatory pathway modulator, an anti-CTLA4 agent, an anti-CTLA4 antibody (human, monoclonal, chimeric, humanized, etc.), ipilimumab, PD1, PDL1, and/or CD137. Additional immunomodulatory agents include, for example, agatolimod, NULOJIX®, blinatumomab, CD40 ligand, anti-B7-1 antibody, anti-B7-2 antibody, anti-B7-H4 antibody, AG4263, eritoran, anti-OX40 antibody, ISF-154, and SGN-70; B7-1, B7-2, ICAM-1, ICAM-2, ICAM-3, CD48, LFA-3, CD30 ligand, CD40 ligand, heat stable antigen, B7h, OX40 ligand, LIGHT, CD70 and CD24.

For the purposes of the present invention, “mammal” refers to humans and other mammals, including, primates, cows, sheep, goats, horses, dogs, cats, rabbits, guinea pigs, rats, mice or other bovine, ovine, equine, canine, feline, rodent or murine species.

In a preferred embodiment of this invention, a method is provided for the synergistic treatment of cancerous tumors. Advantageously, the synergistic method of this invention reduces the development of tumors, reduces tumor burden, or produces tumor regression in a mammalian host.

The combination of a BRAF inhibitor, such as Methyl {5-[2-chloro-2-methylphenyl)-1-hydroxy-3-oxo-2,3-dihydro-1H-isoindol-1-yl]-1H-benzimidazol-2-yl}carbamate with at least one anti-CTLA4 agent, may also include the addition of an anti-proliferative cytotoxic agent either alone or in combination with radiation therapy.

Other anti-proliferative cytotoxic agents are navelbene, CPT-11, anastrazole, letrazole, capecitabine, reloxafine, cyclophosphamide, ifosamide, and droloxafine.

Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as TAXOL®), Mithramycin, Deoxycoformycin, Mitomycin-C, L-Asparaginase, Interferons (especially IFN-a), Etoposide, and Teniposide.

Other combinations with the at least one co-stimulatory pathway modulator, preferably an anti-CTLA4 agent, may include a combination of a co-stimulatory pathway agonist (i.e., immunostimulant), a tubulin stabilizing agent (e.g., pacitaxol, epothilone, taxane, etc.), IXEMPRA®, Dacarbazine, PARAPLATIN®, Docetaxel, one or more peptide vaccines, MDX-1379 Melanoma Peptide Vaccine, one or more gp100 peptide vaccine, fowlpox-PSA-Tricom vaccine, vaccinia-PSA-Tricom vaccine, MART-1 antigen, sargramostim, tremelimumab, Combination Androgen Ablative Therapy; the combination of ipilimumab and another co-stimulatory pathway agonist; combination of ipilimumab and a tubulin stabilizing agent (e.g., pacitaxol, epothilone, taxane, etc.); combination of ipilimumab and IXEMPRA®, the combination of ipilimumab with Dacarbazine, the combination of ipilimumab with PARAPLATIN®, the combination of ipilimumab with Docetaxel, the combination of ipilimumab with one or more peptide vaccines, the combination of ipilimumab with MDX-1379 Melanoma Peptide Vaccine, the combination of ipilimumab with one or more gp100 peptide vaccine, the combination of ipilimumab with fowlpox-PSA-Tricom vaccine, the combination of ipilimumab with vaccinia-PSA-Tricom vaccine, the combination of ipilimumab with MART-1 antigen, the combination of ipilimumab with sargramostim, the combination of ipilimumab with tremelimumab, and/or the combination of ipilimumab with Combination Androgen Ablative Therapy. The combinations of the present invention may also be used in conjunction with other well known therapies that are selected for their particular usefulness against the condition that is being treated.

The phrase “radiation therapy” includes, but is not limited to, x-rays or gamma rays which are delivered from either an externally applied source such as a beam or by implantation of small radioactive sources.

As used in this specification and the appended Claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

BRAF inhibitors, also referred to as Raf inhibitors, known in the art include, for example, compounds of formula I (below) which are described WO 2005/112932, filed Mar. 25, 2005, incorporated herein by reference in its entirety and for all purposes.

Compounds of formula I include the following:

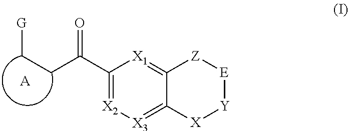

or a pharmaceutically acceptable salt, hydrate or prodrug thereof, wherein:

A is a three- to seven-membered alicyclic, a five- to six-membered ortho-arylene or a five- to six-membered ortho-heteroarylene containing between one and three heteroatoms, either of the aforementioned optionally substituted with up to four R;

each R is independently selected from —H, halogen, —CN, —NO₂, —OR³, —N(R³)R³, —S(O)₀₋₂R³, —SO₂N(R³)R³, —CO₂R³, —C(O)N(R³)R³, —N(R³)SO₂R³, —N(R³)C(O)R³, —N(R³)CO₂R³, —C(O)R³, —OC(O)R³, optionally substituted C₁₋₆alkyl, optionally substituted aryl, optionally substituted aryl C₁₋₆alkyl, optionally substituted heterocyclyl, and optionally substituted heterocyclyl C₁₋₆alkyl;

optionally two of R, together with the atoms to which they are attached, form a first ring system fused with A, said first ring system substituted with zero to three of R¹;

X₁, X₂ and X₃ are independently selected from —CR¹═ or —N═;

each R¹ is independently selected from —H, halogen, —CN, —NO₂, —OR³, —N(R³)R³, —S(O)₀₋₂R³, —SO₂N(R³)R³, —CO₂R³, —C(O)N(R³)R³, —N(R³)SO₂R³, —N(R³)C(O)R³, —N(R³)CO₂R³, —C(O)R³, —OC(O)R³, optionally substituted C₁₋₆alkyl, optionally substituted aryl, optionally substituted aryl C₁₋₆alkyl, optionally substituted heterocyclyl, and optionally substituted heterocyclyl C₁₋₆alkyl;

Z and X are each independently selected from —C(R²)═, —N═, —N(R²)—, —S(O)₀₋₂—, and —O—;

E and Y are each independently selected from absent, —C(R²)(R²)—, —C(═O)—, —C(R²)═ and —N═, but E and Y are not both absent, and E and Y are not both —N═ when both Z and X are —N═;

each R² is independently selected from R³, —N(R³)(R³), —C(O)N(R³)R³, —N(R³)CO₂R³, —N(R³)C(O)N(R³)R³, and —N(R³)C(O)R³;

each R³ is independently selected from —H, optionally substituted C₁₋₆alkyl, optionally substituted C₃₋₇alicyclic, optionally substituted aryl, optionally substituted aryl C₁₋₃alkyl, optionally substituted heterocyclyl, and optionally substituted heterocyclyl C₁₋₃alkyl;

optionally two of R³, when taken together with a common nitrogen to which they are attached, form an optionally substituted five- to seven-membered heterocyclyl, said optionally substituted five- to seven-membered heterocyclyl optionally containing at least one additional heteroatom selected from N, O, S, and P; and

G is selected from —CO₂R³, —C(O)R³, —C(O)N(R³)R³, —C(O)(NR³), —C(O)NR³[C(R³)₂]₀₋₁R³, —C(O)NR³O[C(R³)₂]₀₋₁R³, —N(R³)CO₂R³, —N(R³)C(O)N(R³)R³, —N(R³)C(O)R³, —N(R³)R³, —S(O)₀₋₂R³, SO₂N(R³)R³, optionally substituted aryl C₀₋₃alkyl, and optionally substituted heterocyclyl C₀₋₃alkyl;

with the proviso, however, that the compound is not CAS Registry No. 439096-29-4, 439107-32-1 or 439107-34-3.

One particular example of such a BRAF inhibitor, comprises methyl {5-[2-chloro-2-methylphenyl)-1-hydroxy-3-oxo-2,3-dihydro-1H-isoindol-1-yl]-1H-benzimidazol-2-yl}carbamate, Formula (Ia) (also referred to herein as “Compound Ia”),

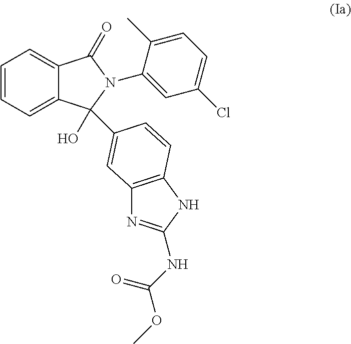 [SMILES: COC(=O)Nc1nc2cc(C3(O)c4ccccc4C(=O)N3c3cc(Cl)ccc3C)ccc2[nH]1]

as described in WO 2005/112932. Methyl {5-[2-chloro-2-methylphenyl)-1-hydroxy-3-oxo-2,3-dihydro-1H-isoindol-1-yl]-1H-benzimidazol-2-yl}carbamate is intended to encompass (unless otherwise indicated) solvates (including hydrates), polymorphic forms of the compound (I) and/or its salts (such as the HCl salt form). Pharmaceutical compositions include all pharmaceutically acceptable compositions comprising methyl {5-[2-chloro-2-methylphenyl)-1-hydroxy-3-oxo-2,3-dihydro-1H-isoindol-1-yl]-1H-benzimidazol-2-yl}carbamate and one or more diluents, vehicles and/or excipients, such as those compositions described in WO 2005/112932.

Another example of a BRAF inhibitor that may be combined with an anti-CTLA4 antagonist include, for example, compounds of formula II (below) which are described in WO2007/013896, filed May 16, 2006, incorporated herein by reference in its entirety and for all purposes. Compounds of Formula II, including all salts, tautomers, and stereoisomers thereof, are encompassed by the present invention and may be included in combination with an anti-CTLA4 antagonist.

Compounds of formula II include the following:

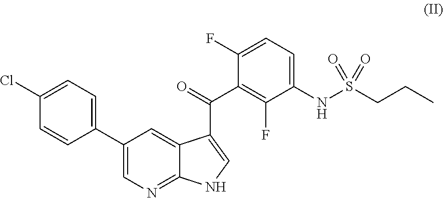 [SMILES: CCCS(=O)(=O)Nc1ccc(F)c(C(=O)c2c[nH]c3ncc(-c4ccc(Cl)cc4)cc23)c1F]

In certain circumstances, it is understood that the use of Compound II may be used in conjunction with a diagnostic test to determine whether or not a patient harbors the V600E Braf mutation. Such a test comprises a method of determining sensitivity of cancer cells to a B-Raf kinase inhibitor, the method comprising: providing a nucleic acid sample from cancer cells from a patient that has a cancer; amplifying a target polynucleotide sequence in the nucleic acid sample using a primer pair that amplifies the target polynucleotide sequence, wherein the target polynucleotide sequence comprises a V600E mutation site in BRAF and amplification is performed in the presence of a labeled oligonucleotide probe that has at least 15 contiguous nucleotides of the canonical encoding sequence of BRAF, and detects the presence of a mutated sequence at the V600E mutation site in BRAF; and detecting the presence or absence of a V600E mutation in BRAF; thereby determining the sensitivity of the cancer to the B-Raf inhibitor. Such a test is disclosed in US2010173294, which published on Jul. 8, 2010, and is incorporated herein by reference in its entirety.

It is to be understood that this invention is not limited to particular methods, reagents, compounds, compositions, or biological systems, which can, of course, vary. It is also to be understood that the terminology used herein is for the purpose of describing particular aspects only, and is not intended to be limiting.

As is known in the art, Ipilimumab refers to an anti-CTLA-4 antibody, and is a fully human IgG_{1κ} antibody derived from transgenic mice having human genes encoding heavy and light chains to generate a functional human repertoire. Ipilimumab can also be referred to by its CAS Registry No. 477202-00-9, and is disclosed as antibody 10DI in PCT Publication No. WO 01/14424, incorporated herein by reference in its entirety and for all purposes. Specifically, Ipilimumab describes a human monoclonal antibody or antigen-binding portion thereof that specifically binds to CTLA4, comprising a light chain variable region and a heavy chain variable region having a light chain variable region comprised of SEQ ID NO:1, and comprising a heavy chain region comprised of SEQ ID NO:2. Pharmaceutical compositions of Ipilimumab include all pharmaceutically acceptable compositions comprising Ipilimumab and one or more diluents, vehicles and/or excipients. Examples of a pharmaceutical composition comprising Ipilimumab are provided in PCT Publication No. WO 2007/67959. Ipilimumab may be administered by I.V.

### Light Chain Variable Region for Ipilimumab:

(SEQ ID NO: 1)
EIVLTQSPGTLSLSPGERATLSCRASQSVGSSYLAWYQQKPGQAPRLLI
YGAFSRATGIPDRFSGSGSGTDFTLTISRLEPEDFAVYYCQQYGSSPWT
FGQGTKVEIK.

### Heavy Chain Variable Region for Ipilimumab:

(SEQ ID NO: 2)
QVQLVESGGGVVQPGRSLRLSCAASGFTFSSYTMHWVRQAPGKGLEWVT
FISYDGNNKYYADSVKGRFTISRDNSKNTLYLQMNSLRAEDTAIYYCAR
TGWLGPFDYWGQGTLVTVSS.

As noted elsewhere herein, the administration of one or more anti-CTLA4 antagonists may be administered either alone or in combination with a peptide antigen (e.g., gp100), in addition to an anti-proliferative agent disclosed herein. A non-limiting example of a peptide antigen would be a gp100 peptide comprising, or alternatively consisting of, the sequence selected from the group consisting of: IMDQVPFSV (SEQ ID NO:3), and YLEPGPVTV (SEQ ID NO:4). Such a peptide may be administered orally, or preferably by injection s.c. at 1 mg emulsified in incomplete Freund's adjuvant (IFA) injected s.c. in one extremity, and 1 mg of either the same or a different peptide emulsified in IFA may be injected in another extremity.

Suitable anti-proliferative agents for use in the methods of the invention, include, without limitation, taxanes, paclitaxel (paclitaxel is commercially available as TAXOL®), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone B1, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, BMS-310705, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

The phrase “microtubulin modulating agent” is meant to refer to agents that either stabilize microtubulin or destabilize microtubulin synthesis and/or polymerization.

As referenced herein, the at least one anti-proliferative agent may be a microtubule affecting agent. A microtubule affecting agent interferes with cellular mitosis and are well known in the art for their anti-proliferative cytotoxic activity. Microtubule affecting agents useful in the invention include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (TAXOL®, NSC 125973), TAXOL® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, epothilone C, epothilone D, desoxyepothilone A, desoxyepothilone B, [1S-[1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo[14.1.0]heptadecane-5,9-dione (disclosed in U.S. Pat. No. 6,262,094, issued Jul. 17, 2001), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Ser. No. 09/506,481 filed on Feb. 17, 2000, and examples 7 and 8 herein), and derivatives thereof; and other microtubule-disruptor agents. Additional antineoplastic agents include, discodermolide (see Service, Science, 274:2009 (1996)) estramustine, nocodazole, MAP4, and the like. Examples of such agents are also described in the scientific and patent literature, see, e.g., Bulinski, J. Cell Sci., 110:3055-3064 (1997); Panda, Proc. Natl. Acad. Sci. USA, 94:10560-10564 (1997); Muhlradt, Cancer Res., 57:3344-3346 (1997); Nicolaou, Nature, 387:268-272 (1997); Vasquez, Mol. Biol. Cell., 8:973-985 (1997); Panda, J. Biol. Chem., 271:29807-29812 (1996).

In cases where it is desirable to render aberrantly proliferative cells quiescent in conjunction with or prior to treatment with the chemotherapeutic methods of the invention, hormones and steroids (including synthetic analogs): 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX® can also be administered to the patient.

Also suitable for use in the combination chemotherapeutic methods of the invention are antiangiogenics such as matrix metalloproteinase inhibitors, and other VEGF inhibitors, such as anti-VEGF antibodies and small molecules such as ZD6474 and SU6668 are also included. Anti-Her2 antibodies from Genentech may also be utilized. A suitable EGFR inhibitor is EKB-569 (an irreversible inhibitor). Also included are Imclone antibody C225 immunospecific for the EGFR, and src inhibitors.

Also suitable for use as an antiproliferative cytostatic agent is CASODEX® which renders androgen-dependent carcinomas non-proliferative. Yet another example of a cytostatic agent is the antiestrogen Tamoxifen which inhibits the proliferation or growth of estrogen dependent breast cancer Inhibitors of the transduction of cellular proliferative signals are cytostatic agents. Examples are epidermal growth factor inhibitors, Her-2 inhibitors, MEK-1 kinase inhibitors, MAPK kinase inhibitors, PI3 inhibitors, Src kinase inhibitors, and PDGF inhibitors.

As mentioned, certain anti-proliferative agents are anti-angiogenic and antivascular agents and, by interrupting blood flow to solid tumors, render cancer cells quiescent by depriving them of nutrition. Castration, which also renders androgen dependent carcinomas non-proliferative, may also be utilized. Starvation by means other than surgical disruption of blood flow is another example of a cytostatic agent. A particularly preferred class of antivascular cytostatic agents is the combretastatins. Other exemplary cytostatic agents include MET kinase inhibitors, MAP kinase inhibitors, inhibitors of non-receptor and receptor tyrosine kinases, inhibitors of integrin signaling, and inhibitors of insulin-like growth factor receptors. The present invention also provides methods for the administration of a protein tyrosine kinase inhibitor, a microtubuline-stabilizing agent, such as paclitaxel; a nucleoside analogue, such as gemcitabine; or a DNA double strand inducing agent, such as etoposide, in synergistic combination(s) with at least one co-stimulatory pathway modulators, particularly an anti-CTLA4 agent, for the treatment and prevention of a proliferative disorder, in addition to a BCR-ABL associated disorder, a mutant BCR-ABL associated disorder, and/or a protein tyrosine kinase-associated disorder, an a disorder associated with the presence of an imatinib-resistant BCR-ABL mutation, a dasatinib-resistant BCR-ABL mutation, CML, imatinib-resistant CML, and/or Imatinib-intolerant CML.

A “solid tumor” includes, for example, sarcoma, melanoma, carcinoma, prostate carcinoma, lung carcinoma, colon carcinoma, or other solid tumor cancer.

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

The synergistic combination of a BRAF inhibitor with a co-stimulatory pathway modulator may also include the addition of one or more additional compounds, which include but are not limited to the following: a tubulin stabilizing agent (e.g., pacitaxol, epothilone, taxane, etc.); a farnysyl transferase inhibitor (e.g., (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt); another protein tyrosine kinase inhibitor; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; the ATP non-competitive inhibitor ONO12380; Aurora kinase inhibitor VX-680; p38 MAP kinase inhibitor BIRB-796; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, or any other combination disclosed herein.

A “farnysyl transferase inhibitor” can be any compound or molecule that inhibits farnysyl transferase. The farnysyl transferase inhibitor can have formula (III), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt. The compound of formula (III) is a cytotoxic FT inhibitor which is known to kill non-proliferating cancer cells preferentially. The compound of formula (III) can further be useful in killing tumor stem cells.

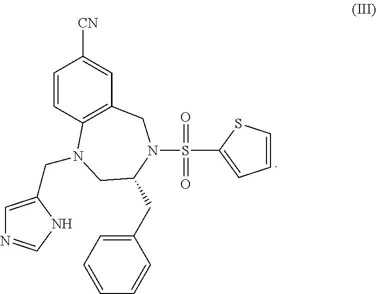 [SMILES: N#Cc1ccc2c(c1)CN(S(=O)(=O)c1cccs1)[C@H](Cc1ccccc1)CN2Cc1cnc[nH]1]

The compound of formula (III), its preparation, and uses thereof are described in U.S. Pat. No. 6,011,029, which is herein incorporated by reference in its entirety and for all purposes. Uses of the compound of formula (III) are also described in WO 2004/015130, published Feb. 19, 2004, which is herein incorporated by reference in its entirety and for all purposes.

The phrase “protein tyrosine kinase” as used herein includes enzymes that catalyze the transfer of the terminal phosphate of adenosine triphosphate (ATP) to tyrosine residues in protein substrates. Non-limiting examples of tyrosine kinases include receptor tyrosine kinases such as EGFR (e.g., EGFR/HER1/ErbB1, HER2/Neu/ErbB2, HER3/ErbB3, HER4/ErbB4), INSR (insulin receptor), IGF-IR, IGF-II1R, IRR (insulin receptor-related receptor), PDGFR (e.g., PDGFRA, PDGFRB), c-KIT/SCFR, VEGFR-1/FLT-1, VEGFR-2/FLK-1/KDR, VEGFR-3/FLT-4, FLT-3/FLK-2, CSF-1R, FGFR 1-4, CCK4, TRK A-C, MET, RON, EPHA 1-8, EPHB 1-6, AXL, MER, TYRO3, TIE, TEK, RYK, DDR 1-2, RET, c-ROS, LTK (leukocyte tyrosine kinase), ALK (anaplastic lymphoma kinase), ROR 1-2, MUSK, AATYK 1-3, and RTK 106; and non-receptor tyrosine kinases such as BCR-ABL, Src, Frk, Btk, Csk, Abl, Zap70, Fes/Fps, Fak, Jak, Ack, and LIMK. One of skill in the art will know of other receptor and/or non-receptor tyrosine kinases that can be targeted using the inhibitors described herein.

The term “tyrosine kinase inhibitor” includes any of a variety of therapeutic agents or drugs that act as selective or non-selective inhibitors of receptor and/or non-receptor tyrosine kinases. Without being bound to any particular theory, tyrosine kinase inhibitors generally inhibit target tyrosine kinases by binding to the ATP-binding site of the enzyme. Examples of tyrosine kinase inhibitors suitable for use in the methods of the present invention include, but are not limited to, PD180970, GGP76030, AP23464, SKI 606, NS-187, AZD0530, gefitinib (IRESSA®), sunitinib (SUTENT®; SU11248), erlotinib (TARCEVA®; OSI-1774), lapatinib (GW572016; GW2016), canertinib (CI-1033), semaxinib (SU5416), vatalanib (PTK787/ZK222584), sorafenib (BAY 43-9006), imatinib (GLEEVEC®; STI571), dasatinib (BMS-354825), leflunomide (SU101), vandetanib (ZACTIMA®; ZD6474), nilotinib, derivatives thereof, analogs thereof, and combinations thereof. Additional tyrosine kinase inhibitors suitable for use in the present invention are described in, e.g., U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340. One of skill in the art will know of other tyrosine kinase inhibitors suitable for use in the present invention

Methods for the safe and effective administration of most of these chemotherapeutic agents are known to those skilled in the art. In addition, their administration is described in the standard literature.

For example, the administration of many of the chemotherapeutic agents is described in the Physicians' Desk Reference (PDR), e.g., 1996 Edition (Medical Economics Company, Montvale, N.J. 07645-1742, USA); the disclosure of which is incorporated herein by reference thereto.

The combination of a chemotherapeutic agent with an immunotherapeutic agent has been previously described. However, the standard dosing regimens have been devoted to administering a chemotherapeutic agent with an immunotherapeutic agent concurrently, but have not previously described the sequential administration of a chemotherapeutic agent followed by of a combination comprising an immunomodulatory agent with a chemotherapeutic agent. In addition, the sequential administration of a chemotherapeutic agent followed by an immunotherapeutic agent has similarly not been described. The present invention supports both of these novel dosing regimens.

For the purposes of the present invention, the sequential administration of one or more cycles of a chemotherapeutic agent followed by one or more cycles of either the combination comprising a chemotherapeutic agent, such as a BRAF inhibitor, and an immunomodulatory agent, or simply an immunomodulatory agent, may optionally comprise an “Intervening Period”, defined as a time period beginning from the end of the last chemotherapeutic cycle up until the beginning of the first immunomodulatory cycle, either concurrently with the last cycle of the chemotherapeutic agent, or sequentially at the end of the one or more chemotherapeutic agent cycle(s). The intervening Period may be about 24 weeks. In another embodiment of the present invention, the intervening Period may be about 20 weeks. In another embodiment of the present invention, the intervening Period may be about 18 weeks. In another embodiment of the present invention, the intervening Period may be about 15 weeks. In another embodiment of the present invention, the intervening Period may be about 12 weeks. In another embodiment of the present invention, the intervening Period may be about 11 weeks. In another embodiment of the present invention, the intervening Period may be about 10 weeks. In another embodiment of the present invention, the intervening Period may be about 9 weeks. In another embodiment of the present invention, the intervening Period may be about 8 weeks. In another embodiment of the present invention, the intervening Period may be about 7 weeks. In another embodiment of the present invention, the intervening Period may be about 6 weeks. In another embodiment of the present invention, the intervening Period may be about 5 weeks. In another embodiment of the present invention, the intervening Period may be about 4 weeks. In another embodiment of the present invention, the intervening Period may be about 3 weeks. In another embodiment of the present invention, the intervening Period may be about 2 weeks. In another embodiment of the present invention, the intervening Period may be about 1 week. In another embodiment of the present invention, the intervening Period may be about 1, 2, 3, 4, 5, 6, or 7 days. In this context, the term “about” shall be construed to mean±1, 2, 3, 4, 5, 6, or 7 days more or less than the stated intervening Period.

In one embodiment of the present invention, the Intervening Period is between 2 to 8 weeks. In another embodiment of the present invention, the Intervening Period is between 3 to 6 weeks.

In another embodiment of the present invention, the Intervening Period may be less than 0 days such that the immunomodulatory agent is administered concurrently with the last cycle of the chemotherapeutic agent.

In another embodiment of the present invention, the Intervening Period may be 0 days such that either the immunomodulatory agent, or a combination comprising an immunomodulatory agent and one or more chemotherapeutic agents, is administered immediately following the last day of the last cycle of the chemotherapeutic agent.

The phrase “immunomodulatory cycle” or “cycle of an immunomodulatory agent” is meant to encompass either one or more dosing cycle(s) of an immunomodulatory agent, or one or more dosing cycle(s) of a combination comprising an immunomodulatory agent and one or more chemotherapeutic agents.

For the purposes of the present invention, “one or more cycles of a chemotherapeutic agent” and/or “one or more cycles of an immunomodulatory agent” means at least 1, at least 2, at least 3, at least 4, at least 5, at least 6, at least 7, at least 8, at least 9, or at least 10 cycles of primary treatment with either agent(s), followed by one or more optional maintenance cycles of either agent(s). The maintenance cycle(s) may follow a similar number of cycles as outlined for the primary therapy, or may be significantly longer or shorter in terms of cycle number, depending upon the patient's disease and/or severity.

In preferred embodiments of the present invention, the phrase “one or more cycles of a chemotherapeutic agent” is meant to encompass one or more cycles of either a chemotherapeutic agent or a combination of one or more chemotherapeutic agents. In one embodiment, “one or more cycles of a chemotherapeutic agent” means more than two cycles, particular of a BRAF inhibitor.

In another aspect of the present invention, the sequential dosing regimen may comprise a “hybrid cycle” in which the patient is administered one or more chemotherapeutic agent cycles, followed by one or more immunomodulatory cycles, followed by one or more chemotherapeutic agent cycles and/or one or more immunomodulatory cycles.

The phrase “sequential dosing regimen”, generally refers to treating a patient with at least two cycles of an agent in a specific order, wherein one cycle is administered after the other. In addition, the phrase “sequential dosing regimen” also encompasses the phrase “phased dosing regimen” as it is traditionally referred to in the pharmaceutical arts. In one context, “sequential dosing regimen” refers to not only the order in which the cycles are administered, but also to the entire treatment regimen for the patient. For example, “sequential dosing regimen” may include the complete dosing regimen for the patient including one or more cycles of a chemotherapeutic agent, followed by one or more cycles of either an immunomodulatory agent or a combination comprising an immunomodulatory agent and one or more chemotherapeutic agents.

For the purposes of the present invention, the sequential administration of a chemotherapeutic agent followed by an immunomodulatory agent, or a combination comprising an immunomodulatory agent and one or more chemotherapeutic agents, is not meant to include the immediate administration of an immunomodulatory agent after failure of an initial chemotherapeutic agent treatment as the cancer patient's primary therapy. Rather, the sequential dosing regimen of the present invention is intended as a stand-alone, primary therapy that includes the sequential administration of a chemotherapeutic agent followed by an immunomodulatory agent, or a combination comprising an immunomodulatory agent and one or more chemotherapeutic agents (i.e., either of which referred to as an “immunomodulatory cycle”). However, the sequential dosing regimen of the present invention may be administered after a sufficient period of time after prior chemotherapeutic therapy has passed, which may be at least about 3 weeks, about 4 weeks, about 5 weeks, about 6 weeks, about 7 weeks, about 8 weeks, about 9 weeks, about 10 weeks, about 11 weeks, about 12 weeks, or more weeks after prior chemotherapeutic therapy has ended and/or after the physician has determined the prior chemotherapeutic therapy had failed.

Additional dosing regimens and therapeutic combinations are disclosed in co-pending provisional application U.S. Ser. No. 61/345,334, filed May 17, 2010, which is hereby incorporated herein in its entirety for all purposes, and in particular dosing regimens and therapeutic combinations.

The compositions of the present invention may further comprise one or more pharmaceutically acceptable additional ingredient(s) such as alum, stabilizers, antimicrobial agents, buffers, coloring agents, flavoring agents, adjuvants, and the like. The pharmaceutical compositions of the present invention may be administered orally or parenterally including the intravenous, intramuscular, intraperitoneal, subcutaneous, rectal and topical routes of administration.

For oral use, the pharmaceutical compositions of the present invention, may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The co-stimulatory pathway modulator, preferably an anti-CTLA4 agent, described herein may also be delivered transdermally. The transdermal compositions can take the form of creams, lotions, aerosols and/or emulsions and can be included in a transdermal patch of the matrix or reservoir type as are conventional in the art for this purpose.

If formulated as a fixed dose, the active ingredients of the pharmaceutical combination compositions of the present invention are employed within the dosage ranges described below. Alternatively, the co-stimulatory pathway modulator and the protein tyrosine kinase inhibitor may be administered separately in the dosage ranges described below. In a preferred embodiment of the present invention, the co-stimulatory pathway modulator is administered in the dosage range described below following or simultaneously with administration of the protein tyrosine kinase inhibitor in the dosage range described below.

The following sets forth preferred therapeutic combinations and exemplary dosages for use in the methods of the present invention.

| | DOSAGE |
|---|---|
| THERAPEUTIC COMBINATION | mg/m² (per dose)¹ |
| First Administration of Methyl {5-[2- | 5-180 mg PD or BID |
| chloro-2-methylphenyl)-1-hydroxy-3-oxo-2,3- | 0.1-25 mg/kg |
| dihydro-1H-isoindol-1-yl]-1H-benzimidazol- | |
| 2-yl} carbamate, with Administration of | |
| anti-CTLA4 Antibody | |

¹Each combination listed herein optionally includes the administration of an anti-cancer vaccine from about 0.001-100 mg.

While this table provides exemplary dosage ranges of the BRAF inhibitor, preferably Methyl {5-[2-chloro-2-methylphenyl)-1-hydroxy-3-oxo-2,3-dihydro-1H-isoindol-1-yl]-1H-benzimidazol-2-yl}carbamate, a co-stimulator pathway modulator, preferably anti-CTLA4 antibody, and/or anti-cancer vaccine agents, when formulating the pharmaceutical compositions of the invention the clinician may utilize preferred dosages as warranted by the condition of the patient being treated. The anti-CTLA4 antibody may preferably be administered at about 0.3-10 mg/kg, or the maximum tolerated dose. In an embodiment of the invention, a dosage of CTLA-4 antibody is administered about every three weeks. Alternatively, the CTLA-4 antibody may be administered by an escalating dosage regimen including administering a first dosage of CTLA-4 antibody at about 3 mg/kg, a second dosage of CTLA-4 antibody at about 5 mg/kg, and a third dosage of CTLA-4 antibody at about 9 mg/kg.

Likewise, the BRAF inhibitor, preferably Methyl {5-[2-chloro-2-methylphenyl)-1-hydroxy-3-oxo-2,3-dihydro-1H-isoindol-1-yl]-1H-benzimidazol-2-yl}carbamate, may preferably be administered once per day at about 5, about 25, about 100, or about 150 mg per day; at about 2 times per day at about 5, about 25, about 100, or about 150 mg. Alternatively, it can be dosed at, for example, about 5, about 10, about 15, about 20, about 25, about 30, about 35, about 40, about 50, about 70, about 90, about 100, 110, or 120 per day; or about 5, about 10, about 15, about 20, about 25, about 30, about 35, about 40, about 50, about 70, about 90, about 100, 110, or 120 twice per day, or the maximum tolerated dose. The dose of a BRAF inhibitor may depend upon a number of factors, including stage of disease, the presence of one or more mutations in the targeted BRAF kinase, etc. The specific dose that should be administered based upon the presence of one or more of such factors is within the skill of the artisan.

The combinations of the present invention may also be used in conjunction with other well known therapies that are selected for their particular usefulness against the condition that is being treated.

The anti-CTLA4 antibody may preferably be administered at about 0.3-10 mg/kg, or the maximum tolerated dose. In an embodiment of the invention, a dosage of CTLA-4 antibody is administered about every three weeks. Alternatively, the CTLA-4 antibody may be administered by an escalating dosage regimen including administering a first dosage of CTLA-4 antibody at about 3 mg/kg, a second dosage of CTLA-4 antibody at about 5 mg/kg, and a third dosage of CTLA-4 antibody at about 9 mg/kg.

In another specific embodiment, the escalating dosage regimen includes administering a first dosage of CTLA-4 antibody at about 5 mg/kg and a second dosage of CTLA-4 antibody at about 9 mg/kg.

Further, the present invention provides an escalating dosage regimen, which includes administering an increasing dosage of CTLA-4 antibody about every six weeks.

In an aspect of the present invention, a stepwise escalating dosage regimen is provided, which includes administering a first CTLA-4 antibody dosage of about 3 mg/kg, a second CTLA-4 antibody dosage of about 3 mg/kg, a third CTLA-4 antibody dosage of about 5 mg/kg, a fourth CTLA-4 antibody dosage of about 5 mg/kg, and a fifth CTLA-4 antibody dosage of about 9 mg/kg. In another aspect of the present invention, a stepwise escalating dosage regimen is provided, which includes administering a first dosage of 5 mg/kg, a second dosage of 5 mg/kg, and a third dosage of 9 mg/kg.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

When employing the methods or compositions of the present invention, other agents used in the modulation of tumor growth or metastasis in a clinical setting, such as antiemetics, can also be administered as desired.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-CTLA4 agent(s) and protein tyrosine kinase inhibitor) on the patient, and in view of the observed responses of the disease to the administered therapeutic agents.

In the methods of this invention, a BRAF inhibitor, such as Methyl {5-[2-chloro-2-methylphenyl)-1-hydroxy-3-oxo-2,3-dihydro-1H-isoindol-1-yl]-1H-benzimidazol-2-yl}carbamate, is administered simultaneously or sequentially with an anti-CTLA4 agent. Thus, it is not necessary that the anti-CTLA4 therapeutic agent(s) and a BRAF inhibitor be administered simultaneously or essentially simultaneously. The advantage of a simultaneous or essentially simultaneous administration is well within the determination of the skilled clinician.

Also, in general, a BRAF inhibitor and anti-CTLA4 agent(s) do not have to be administered in the same pharmaceutical composition, and may, because of different physical and chemical characteristics, have to be administered by different routes.

If a BRAF inhibitor and the anti-CTLA4 agent(s) are not administered simultaneously or essentially simultaneously, then the initial order of administration of a BRAF inhibitor and the anti-CTLA4 agent(s) may be varied. Thus, for example, the BRAF inhibitor Methyl {5-[2-chloro-2-methylphenyl)-1-hydroxy-3-oxo-2,3-dihydro-1H-isoindol-1-yl]-1H-benzimidazol-2-yl}carbamate, for example, may be administered first followed by the administration of the anti-CTLA4 agent(s); or the anti-CTLA4 agent(s) may be administered first followed by the administration of a BRAF inhibitor. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient.

Thus, in accordance with experience and knowledge, the practicing physician can modify each protocol for the administration of a component (therapeutic agent—i.e., a BRAF inhibitor, anti-CTLA4 agent(s)) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

As referenced elsewhere herein, the optimal dose for the BRAF inhibitor may depend upon a number of factors.

### Additional Anti-CTLA4 Compositions

The present invention also encompasses additional anti-CTLA-4 agents including, but not limited to, an anti-CTLA-4 antibody, an anti-CTLA-4 adnectin, an anti-CTLA-4 RNAi, single chain anti-CTLA-4 antibody fragments, domain anti-CTLA-4 antibody fragments, and an anti-CTLA-4 antisense molecule.

A preferred anti-CTLA4 agent of the present invention is the anti-CTLA4 antibody ipilimumab. Other anti-CTLA4 antibodies and fragments are encompassed by the present invention which immunospecifically bind a polypeptide, polypeptide fragment, or variant of CTLA4, and/or an epitope of CTLA4 (as determined by immunoassays well known in the art for assaying specific antibody-antigen binding). Antibodies include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody”, as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F(ab′)2 fragments) which are capable of specifically binding to protein. Fab and F(ab′)2 fragments lack the Fc fragment of intact antibody, clear more rapidly from the circulation of the animal or plant, and may have less non-specific tissue binding than an intact antibody (Wahl et al., J. Nucl. Med., 24:316-325 (1983)). Thus, these fragments are preferred, as well as the products of a FAB or other immunoglobulin expression library. Moreover, anti-CTLA4 antibodies include chimeric, single chain, and humanized antibodies.

The anti-CTLA4 antibodies can be produced by any method known in the art for the synthesis of antibodies, in particular, by chemical synthesis or preferably, by recombinant expression techniques.

The adnectins of the present invention may be made according to the methods outlined in co-owned U.S. Publication Nos. 2007/0082365, and 2008/0139791.

Techniques described for the production of single chain antibodies (U.S. Pat. No. 4,946,778; Bird, Science, 242:423-442 (1988); Huston et al., Proc. Natl. Acad. Sci. USA, 85:5879-5883 (1988); and Ward et al., Nature, 334:544-554 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science, 242:1038-1041 (1988)).

Recombinant expression of an anti-CTLA4 antibody, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an anti-CTLA4 antibody molecule or a heavy or light chain of an antibody, or portion thereof (preferably containing the heavy or light chain variable domain), has been obtained, the vector for the production of the anti-CTLA4 antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an anti-CTLA4 antibody, or a heavy or light chain thereof, or a heavy or light chain variable domain, operably linked to a promoter. Such vectors may include the nucleotide sequence encoding the constant region of the antibody molecule (see, e.g., PCT Publication Nos. WO 86/05807 and WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an anti-CTLA4 antibody. Thus, the invention includes host cells containing a polynucleotide encoding an anti-CTLA4 antibody, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the anti-CTLA4 antibody molecules. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene, 45:101 (1986); Cockett et al., Bio/Technology, 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J., 2:1791 (1983)), in which the antibody coding sequence may be ligated individually into the vector in frame with the lac Z coding region so that a fusion protein is produced; pIN vectors (Inouye et al., Nucleic Acids Res., 13:3101-3109 (1985); Van Heeke et al., J. Biol. Chem., 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the anti-CTLA4 antibody coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan et al., Proc. Natl. Acad. Sci. USA, 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al., Meth. Enzymol., 153:516-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the anti-CTLA4 antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the anti-CTLA4 antibody molecule.

A number of selection systems may be used, including but not limited to the herpes simplex virus thymidine kinase (Wigler et al., Cell, 11:223 (1977)), hypoxanthine-guanine phosphoribosyltransferase (Szybalska et al., Proc. Natl. Acad. Sci. USA, 48:202 (1992)), and adenine phosphoribosyltransferase (Lowy et al., Cell, 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Proc. Natl. Acad. Sci. USA, 77:357 (1980); O'Hare et al., Proc. Natl. Acad. Sci. USA, 78:1527 (1981)); gpt, which confers resistance to mycophenolic acid (Mulligan et al., Proc. Natl. Acad. Sci. USA, 78:2072 (1981)); neo, which confers resistance to the aminoglycoside G-418, Clinical Pharmacy, 12(7):488-505 (1993); Wu et al., Biotherapy, 3:87-95 (1991); Tolstoshev, Ann. Rev. Pharmacol. Toxicol., 32:573-596 (1993); Mulligan, Science, 260:926-932 (1993); and Morgan et al., Ann. Rev. Biochem., 62:191-217 (1993); TIB TECH, 11(5):155-215 (May 1993)); and hygro, which confers resistance to hygromycin (Santerre et al., Gene, 30:147 (1984)). Methods commonly known in the art of recombinant DNA technology may be routinely applied to select the desired recombinant clone, and such methods are described, for example, in Ausubel et al., eds., Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al., eds., Current Protocols in Human Genetics, John Wiley & Sons, NY (1994); Colberre-Garapin et al., J. Mol. Biol., 150:1 (1981), which are incorporated by reference herein in their entireties.

The expression levels of an anti-CTLA4 antibody molecule can be increased by vector amplification (for a review, see Bebbington et al., “The use of vectors based on gene amplification for the expression of cloned genes in mammalian cells” in DNA Cloning, Vol. 3, Academic Press, NY (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol., 3:257 (1983)).

The host cell may be co-transfected with two expression vectors, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 322:52 (1986); Kohler, Proc. Natl. Acad. Sci. USA, 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the anti-CTLA4 antibodies or fragments thereof can be fused to heterologous polypeptide sequences described herein or otherwise known in the art, to facilitate purification.

The present invention further includes compositions comprising polypeptides or conjugated to anti-CTLA4 antibody domains other than the variable regions. For example, the polypeptides may be fused or conjugated to an antibody Fc region, or portion thereof. The anti-CTLA4 antibody portion fused to a polypeptide may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603, 5,622,929, 5,359,046, 5,349,053, 5,447,851, and 5,112,946; EP 307,434; EP 367,166; PCT Publication Nos. WO 96/04388 and WO 91/06570; Ashkenazi et al., Proc. Natl. Acad. Sci. USA, 88:10535-10539 (1991); Zheng et al., J. Immunol., 154:5590-5600 (1995); and Vil et al., Proc. Natl. Acad. Sci. USA, 89:11337-11341 (1992) (said references incorporated by reference in their entireties).

Further, an anti-CTLA4 antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, an apoptotic agent, e.g., TNF-alpha, TNF-beta, AIM I (See PCT Publication No. WO 97/33899), AIM II (See PCT Publication No. WO 97/34911), Fas Ligand (Takahashi et al., Int. Immunol., 6:1567-1574 (1994)), VEGI (See PCT Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies for Immunotargeting of Drugs in Cancer Therapy”, in Monoclonal Antibodies and Cancer Therapy, Reisfeld et al., eds., pp. 243-256, Alan R. Liss, Inc. (1985); Hellstrom et al., “Antibodies for Drug Delivery”, in Controlled Drug Delivery, 2nd Edition, Robinson et al., eds., pp. 623-653, Marcel Dekker, Inc. (1987); Thorpe, “Antibody Carriers of Cytotoxic Agents in Cancer Therapy: A Review”, in Monoclonal Antibodies '84: Biological and Clinical Applications, Pinchera et al., eds., pp. 475-506 (1985); “Analysis, Results, and Future Prospective of the Therapeutic Use of Radiolabeled Antibody in Cancer Therapy”, in Monoclonal Antibodies for Cancer Detection and Therapy, Baldwin et al., eds., pp. 303-316, Academic Press (1985), and Thorpe et al., “The Preparation and Cytotoxic Properties of Antibody-Toxin Conjugates”, Immunol. Rev., 62:119-158 (1982).

Alternatively, an anti-CTLA4 antibody can be conjugated to a second antibody to form an antibody heteroconjugate as described by Segal in U.S. Pat. No. 4,676,980, which is incorporated herein by reference in its entirety.

An anti-CTLA4 antibody, with or without a therapeutic moiety conjugated to it, administered alone or in combination with cytotoxic factor(s) and/or cytokine(s) can be used as a therapeutic.

The present invention also encompasses the creation of synthetic antibodies directed against the polypeptides of the present invention. One example of synthetic antibodies is described in Radrizzani, M. et al., Medicina (Aires), 59(6):753-758 (1999)). Recently, a new class of synthetic antibodies has been described and are referred to as molecularly imprinted polymers (MIPs) (Semorex, Inc.). Antibodies, peptides, and enzymes are often used as molecular recognition elements in chemical and biological sensors. However, their lack of stability and signal transduction mechanisms limits their use as sensing devices. Molecularly imprinted polymers (MIPs) are capable of mimicking the function of biological receptors but with less stability constraints. Such polymers provide high sensitivity and selectivity while maintaining excellent thermal and mechanical stability. MIPs have the ability to bind to small molecules and to target molecules such as organics and proteins with equal or greater potency than that of natural antibodies. These “super” MIPs have higher affinities for their target and thus require lower concentrations for efficacious binding.

During synthesis, the MIPs are imprinted so as to have complementary size, shape, charge and functional groups of the selected target by using the target molecule itself (such as a polypeptide, antibody, etc.), or a substance having a very similar structure, as its “print” or “template”. MIPs can be derivatized with the same reagents afforded to antibodies. For example, fluorescent ‘super’ MIPs can be coated onto beads or wells for use in highly sensitive separations or assays, or for use in high throughput screening of proteins.

A number of methods may be employed to create MIPs to a specific receptor, ligand, polypeptide, peptide, organic molecule. Several preferred methods are described by Esteban et al. in J. Anal. Chem., 370(7):795-802 (2001), which is hereby incorporated herein by reference in its entirety in addition to any references cited therein. Additional methods are known in the art and are encompassed by the present invention, such as for example, Hart, B. R. et al., J. Am. Chem. Soc., 123(9):2072-2073 (2001); and Quaglia, M. et al., J. Am. Chem. Soc., 123(10):2146-2154 (2001); which are hereby incorporated by reference in their entirety herein.

Antisense oligonucleotides may be single or double stranded. Double stranded RNA's may be designed based upon the teachings of Paddison et al., Proc. Nat. Acad. Sci., 99:1443-1448 (2002); and PCT Publication Nos. WO 01/29058, and WO 99/32619; which are hereby incorporated herein by reference.

Double stranded RNA may also take the form of an RNA inhibitor (“RNAi”) such that they are competent for RNA interference. For example, anti-CTLA4 RNAi molecules may take the form of the molecules described by Mello and Fire in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438. The teachings of these patent and patent applications are hereby incorporated herein by reference in their entirety.

For example, the anti-CTLA4 RNAi molecules may be double stranded RNA, and between about 25 to 400 nucleotides in length, and complementary to the encoding nucleotide sequence of CTLA4. Such RNAi molecules may be about 20, about 25, about 30, about 35, about 45, and about 50 nucleotides in length. In this context, the term “about” is construed to be about 1, 2, 3, 4, 5, or 6 nucleotides longer in either the 5′ or 3′ direction, or both.

Alternatively, the anti-CTLA4 RNAi molecules of the present invention may take the form be double stranded RNAi molecules described by Kreutzer in European Patent Nos. EP 1144639, and EP 1214945. The teachings of these patent and patent applications are hereby incorporated herein by reference in their entirety. Specifically, the anti-CTLA4 RNAi molecules of the present invention may be double stranded RNA that is complementary to the coding region of CTLA4, and is between about 15 to about 49 nucleotides in length, and preferably between about 15 to about 21 nucleotides in length. In this context, the term “about” is construed to be about 1, 2, 3, 4, 5, 6, 7, 8, 9, or 10 nucleotides longer in either the 5′ or 3′ direction, or both. Such anti-CTLA-4 molecules can be stabilized by chemical linkage of the single RNA strands.

Alternatively, the anti-CTLA4 RNAi molecules of the present invention may take the form be double stranded RNAi molecules described by Tuschl in European Patent No. EP 1309726. The teachings of these patent and patent applications are hereby incorporated herein by reference in their entirety. Specifically, the anti-CTLA4 RNAi molecules of the present invention may be double stranded RNA that is complementary to the coding region of CTLA4, and is between about 21 to about 23 nucleotides in length, and are either blunt ended or contain either one or more overhangs on the 5′ end or 3′ end of one or both of the strands with each overhang being about 1, 2, 3, 4, 5, 6, or more nucleotides in length. The ends of each strand may be modified by phosphorylation, hydroxylation, or other modifications. In addition, the internucleotide linkages of one or more of the nucleotides may be modified, and may contain 2′-OH. In this context, the term “about” is construed to be about 1, 2, 3, 4, 5, 6, 7, 8, 9, or 10 nucleotides longer in either the 5′ or 3′ direction, or both. Such anti-CTLA-4 molecules can be stabilized by chemical linkage of the single RNA strands.

Alternatively, the anti-CTLA4 RNAi molecules of the present invention may take the form be double stranded RNAi molecules described by Tuschl in U.S. Pat. Nos. 7,056,704 and 7,078,196. The teachings of these patent and patent applications are hereby incorporated herein by reference in their entirety. Specifically, the anti-CTLA4 RNAi molecules of the present invention may be double stranded RNA that is complementary to the coding region of CTLA4, and is between about 19 to about 25 nucleotides in length, and are either blunt ended or contain either one or more overhangs on the 5′ end or 3′ end of one or both of the strands with each overhang being about 1, 2, 3, 4, or 5 or more nucleotides in length. The ends of each strand may be modified by phosphorylation, hydroxylation, or other modifications. In addition, the internucleotide linkages of one or more of the nucleotides may be modified, and may contain 2′-OH. In this context, the term “about” is construed to be about 1, 2, 3, 4, 5, 6, 7, 8, 9, or 10 nucleotides longer in either the 5′ or 3′ direction, or both. Such anti-CTLA-4 molecules can be stabilized by chemical linkage of the single RNA strands.

Additionally, the anti-CTLA4 RNAi molecules of the present invention may take the form be RNA molecules described by Crooke in U.S. Pat. Nos. 5,898,031, 6,107,094, 7,432,249, and 7,432,250, and European Application No. EP 0928290. The teachings of these patent and patent applications are hereby incorporated herein by reference in their entirety. Specifically, the anti-CTLA4 molecules may be single stranded RNA, containing a first segment having at least one ribofuranosyl nucleoside subunit which is modified to improve the binding affinity of said compound to the preselected RNA target when compared to the binding affinity of an unmodified oligoribonucleotide to the RNA target; and a second segment comprising at least four consecutive ribofuranosyl nucleoside subunits having 2′-hydroxyl moieties thereon; said nucleoside subunits of said oligomeric compound being connected by internucleoside linkages which are modified to stabilize said linkages from degradation as compared to phosphodiester linkages. Preferably, such RNA molecules are about 15 to 25 nucleotides in length, or about 17 to about 20 nucleotides in length. Preferably such molecules are competent to activate a double-stranded RNAse enzyme to effect cleavage of CTLA4 RNA. In this context, the term “about” is construed to be about 1, 2, 3, 4, 5, 6, 7, 8, 9, or 10 nucleotides longer in either the 5′ or 3′ direction, or both. Such anti-CTLA-4 molecules can be stabilized by chemical linkage of the single RNA strands.

SiRNA reagents are specifically contemplated by the present invention. Such reagents are useful for inhibiting expression of the polynucleotides of the present invention and may have therapeutic efficacy. Several methods are known in the art for the therapeutic treatment of disorders by the administration of siRNA reagents. One such method is described by Tiscornia et al. (Proc. Natl. Acad. Sci., 100(4):1844-1848 (2003)); WO 04/09769, filed Jul. 18, 2003; and Reich, S. J. et al., Mol. Vis., 9:210-216 (May 30, 2003), which are incorporated by reference herein in its entirety.

In order to facilitate a further understanding of the invention, the following examples are presented primarily for the purpose of illustrating more specific details thereof. The scope of the invention should not be deemed limited by the examples, but to encompass the entire subject matter defined by the Claims.

### REFERENCES

Heidorn, S. J. et al., “Kinase-dead BRAF and oncogenic RAS cooperate to drive tumour progression through CRAF,” Cell, 140:209-221 (2010).
Poulikakos, P. I. et al., “RAF inhibitors transactivate RAF dimers and ERK signalling in cells with wild-type BRAF,” Nature (23 Feb. 2010).
Hatzivassiliou, G. et al., “RAF inhibitors prime wild-type RAF to activate the MAPK pathway and enhance growth,” Nature (3 Feb. 2010).

### EXAMPLES

### Example 1

### Method of Assessing the Effect of the Combination of a BRAF Inhibitor with a Co-Stimulatory Pathway Modulator on Tumor Growth in a Murine Colon Cancer Tumor Model

The Ras-Raf-MEK-ERK pathway may be constitutively activated in human cancers through mutations in Ras or Raf (Halilovic et al., McCubrey et al.; and Michaloglou et al.). Based on its association with human cancers, BRAF has been a target for therapeutic treatment of cancer.

Thus, there was an interest in determining whether a potentiation of an antitumor immune response could be achieved by the combination of a CTLA-4 blocking mAb and a BRAF inhibitor in a murine CT26 colon carcinoma model.

Pharmacology studies are currently being conducted in support of the proposed clinical trial evaluating the antitumor activity of the BRAF inhibitor Methyl {5-[2-chloro-2-methylphenyl)-1-hydroxy-3-oxo-2,3-dihydro-1H-isoindol-1-yl]-1H-benzimidazol-2-yl}carbamate, and ipilimumab in subjects with advanced melanoma. Efficacy studies were conducted in the CT26 colon carcinoma, a tumor model sensitive to CTLA-4 blockade.

Two different combination schedules were evaluated, concurrent and sequential. In the concurrent setting, Methyl {5-[2-chloro-2-methylphenyl)-1-hydroxy-3-oxo-2,3-dihydro-1H-isoindol-1-yl]-1H-benzimidazol-2-yl}carbamate (100 mg/kg, p.o.) was administered every other day for 7 doses from day 4-14 while CTLA-4 mAb (20 mg/kg, i.p.) was dosed every 4 days for 3 doses starting on day 5. In the sequential setting, Methyl {5-[2-chloro-2-methylphenyl)-1-hydroxy-3-oxo-2,3-dihydro-1H-isoindol-1-yl]-1H-benzimidazol-2-yl}carbamate (100 mg/kg, p.o.) was dosed every other day for 4 doses from day 4-10 while CTLA-4 mAb (20 mg/kg, i.p.) was dosed every 4 days for 3 doses starting on day 11. Control groups consisted of each agent dosed in combination with vehicle control following the same schedule.

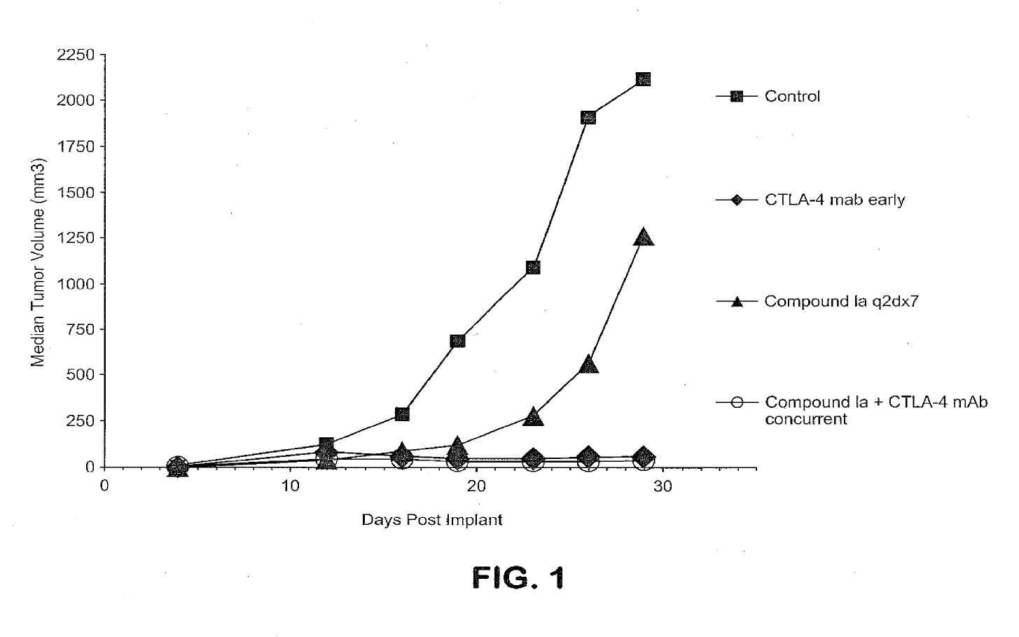

As shown in FIG. 1, concurrent administration of Methyl {5-[2-chloro-2-methylphenyl)-1-hydroxy-3-oxo-2,3-dihydro-1H-isoindol-1-yl]-1H-benzimidazol-2-yl}carbamate and CTLA-4 mAb resulted in an antitumor effect similar to CTLA-4 mAb alone (% TGI of 98 and 97 respectively, Day 26) while Methyl {5-[2-chloro-2-methylphenyl)-1-hydroxy-3-oxo-2,3-dihydro-1H-isoindol-1-yl]-1H-benzimidazol-2-yl}carbamate inhibited tumor growth by 70%.

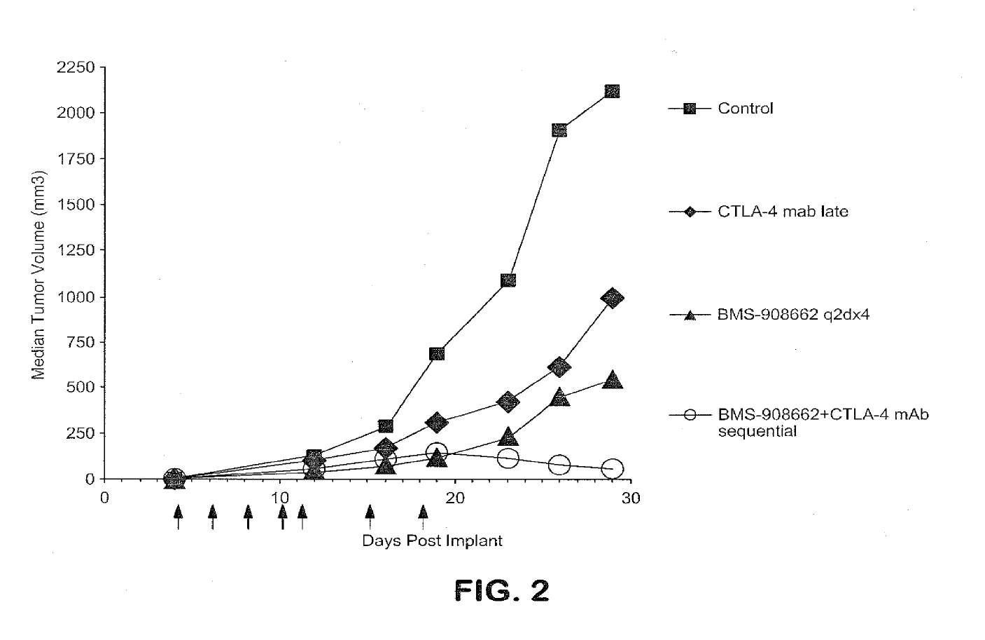

Surprisingly, the sequential administration of Methyl {5-[2-chloro-2-methylphenyl)-1-hydroxy-3-oxo-2,3-dihydro-1H-isoindol-1-yl]-1H-benzimidazol-2-yl}carbamate followed by CTLA-4 mAb showed enhanced antitumor activity compared to CTLA-4 mAb alone as shown in FIG. 2. Specifically, when these agents were administered sequentially, the % TGI on day 26 was 96% for the combination group, 77% for Methyl {5-[2-chloro-2-methylphenyl)-1-hydroxy-3-oxo-2,3-dihydro-1H-isoindol-1-yl]-1H-benzimidazol-2-yl}carbamate and 68% for CTLA-4 mAb (FIG. 2). In this setting, the activity of CTLA-4 mAb was reduced compared to the study shown in FIG. 1 because treatments were initiated when tumors were established (average size=100 mm³).

Results from these studies suggest that concurrent administration of Methyl {5-[2-chloro-2-methylphenyl)-1-hydroxy-3-oxo-2,3-dihydro-1H-isoindol-1-yl]-1H-benzimidazol-2-yl}carbamate and CTLA-4 mAb did not affect the activity elicited by either agent as monotherapy, with the caveat that CTLA-4 mAb produced complete inhibition of tumor growth when dosing was initiated at early time points. Furthermore, an antitumor effect superior to each agent alone was observed when Methyl {5-[2-chloro-2-methylphenyl)-1-hydroxy-3-oxo-2,3-dihydro-1H-isoindol-1-yl]-1H-benzimidazol-2-yl}carbamate was dosed prior to CTLA-4 mAb. The latter result was observed in a setting suboptimal for anti-CTLA-4 activity, with CTLA-4 treatment initiated on day 11 compared to initial dosing on day 5 in the concurrent study. Studies continue to be monitored to assess the effect of these treatment on tumor regressions.

In summary, synergistic effects were observed with concurrent treatment and sequential treatment with CTLA-4 mAb+BRAF inhibitor, with the sequential treatment showing surprising, enhanced response in the CT26 colon tumor model as shown in FIGS. 1 and 2.

### Example 2

### Method of Assessing the Effect of the Combination of a BRAF Inhibitor with a Co-Stimulatory Pathway Modulator on Tumor Growth in a Murine Antigen-Induced T-Cell Proliferation Model

The effect of the combination of a BRAF inhibitor and CTLA-4 mAb was evaluated in a model of antigen-induced T-cell proliferation. In this model, T cells specific for the antigen ovalbumin (OVA) were adoptively transferred into naive C57BL6 mice on study day −1. On Study Day 0, animals were challenged with 250 ug of an OVA peptide (i.v.). Animals were reandomized and divided into groups of 5. Treatments were administered as follows: CTLA-4 mAb—20 mg/kg—study days 1 and 3; Methyl {5-[2-chloro-2-methylphenyl)-1-hydroxy-3-oxo-2,3-dihydro-1H-isoindol-1-yl]-1H-benzimidazol-2-yl}carbamate—100 mg/kg—study days 1, 3, and 5. In addition, another study group received erlotinib (TARCEVA®) daily at 100 mg/kg, p.o. from study days 1 through 5. Combination groups consisted of Compound Ia and CTLA-4 mAb or erlotinib+CTLA-4 mAb following the same schedules. Control animals received the OVA challenge and no further treatment. Expansion of OVA-specific T cells was measured prior to OVA challenge and on days 2, 5, 7 and 16 by OVA-specific tetramer staining.

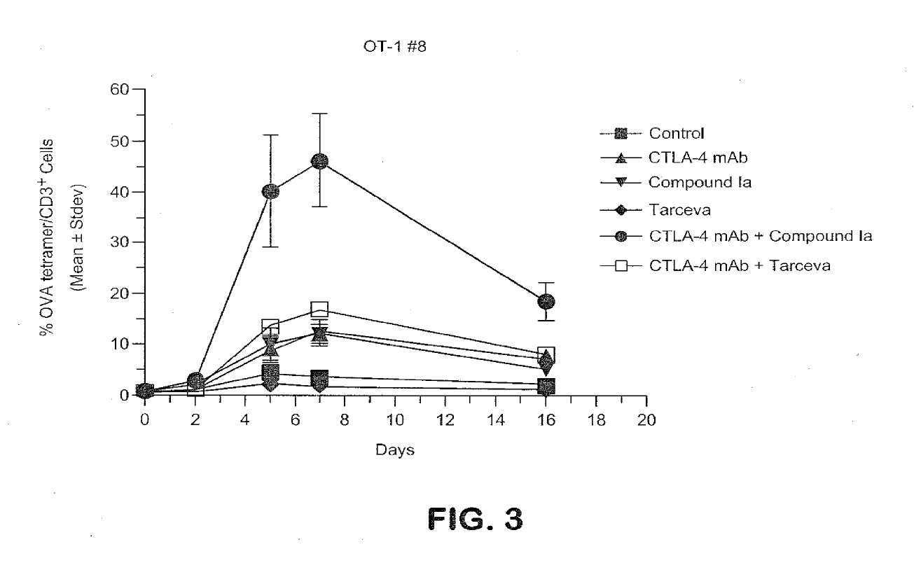

As shown in FIG. 3, as expected, CTLA-4 mAb significantly increased OVA tetramer response vs. control (p<0.05). Unexpectedly, Methyl {5-[2-chloro-2-methylphenyl)-1-hydroxy-3-oxo-2,3-dihydro-1H-isoindol-1-yl]-1H-benzimidazol-2-yl}carbamate showed an effect similar to CTLA-4 mAb, while erlotinib decreased the response. However, erlotinib did not abrogate CTLA-4 mAb effect when it was administered in combination with CTLA-4 mAb since the response was similar to CTLA-4 mAb alone. Surprisingly, CTLA-4 mAb+Methyl {5-[2-chloro-2-methylphenyl)-1-hydroxy-3-oxo-2,3-dihydro-1H-isoindol-1-yl]-1H-benzimidazol-2-yl}carbamate synergistically increased OVA tetramer response on days 5, 7 and 16 (synergy=effect of the combination was significantly superior (P<0.05) than the effect of each agent alone).

In summary, synergistic effects were observed with the concurrent treatment of CTLA-4 mAb+BRAF inhibitor in an antigen ovalbumin (OVA) model of antigen-induced T-cell proliferation, as shown in FIGS. 1 and 2.

These results were consistent with the results observed in the CT26 tumor model (see FIGS. 1 and 2), and confirms the administration of a BRAF inhibitor in combination with a CTLA-4 antibody results in synergistic inhibition in tumor proliferation.

### Example 3

### Method of Assessing the Effect of the Combination of a BRAF Inhibitor with a Co-Stimulatory Pathway Modulator on Tumor Growth in a Murine Sa1N Tumor Model

Previously, the combination of CTLA-4 mAb with Methyl {5-[2-chloro-2-methylphenyl)-1-hydroxy-3-oxo-2,3-dihydro-1H-isoindol-1-yl]-1H-benzimidazol-2-yl}carbamate produced superior antitumor activity compared to each agent alone in the CT26 colon carcinoma model. In addition, combination of both agents synergistically expanded antigen-specific T cells when dosed concurrently. The effect of this promising combination was further investigated in a) a tumor model sensitive to CTLA-4 blockade (SA1N fibrosarcoma); b) in the antigen (OVA)-specific T cell model to assess the effect of Methyl {5-[2-chloro-2-methylphenyl)-1-hydroxy-3-oxo-2,3-dihydro-1H-isoindol-1-yl]-1H-benzimidazol-2-yl}carbamate at different dose levels and compared its activity to another BRAF inhibitor (EXT-000153, Genentech) and EVRI.

In the SA1N tumor model, the effect of the combination of both agents was investigated under either a concurrent dosing regimen or a sequential schedule. Only results from sequential treatment are shown, because extended dosing with Methyl {5-[2-chloro-2-methylphenyl)-1-hydroxy-3-oxo-2,3-dihydro-1H-isoindol-1-yl]-1H-benzimidazol-2-yl}carbamate (7 doses used in the concurrent regimen) alone or in combination with CTLA-4 mAb was toxic.

Sa1N tumors were implanted s.c. and treatments were administered as follows: Methyl {5-[2-chloro-2-methylphenyl)-1-hydroxy-3-oxo-2,3-dihydro-1H-isoindol-1-yl]-1H-benzimidazol-2-yl}carbamate, 100 mg/kg, Q2D×4 starting on day 10 CTLA-4 mAb, 20 mg/kg, Q4D×3, starting on day 17.

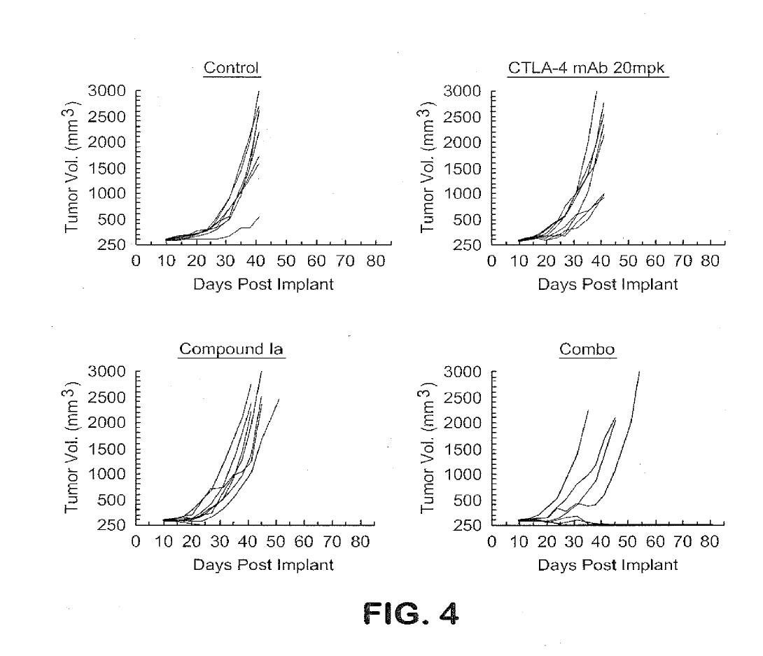

As shown in FIG. 4, when mice were dosed with the combination of Methyl {5-[2-chloro-2-methylphenyl)-1-hydroxy-3-oxo-2,3-dihydro-1H-isoindol-1-yl]-1H-benzimidazol-2-yl}carbamate and CTLA-4 mAb, a surprising, enhanced antitumor effect was observed compared with the effect elicited by each treatment alone. While no regression were observed with these agents as monotherapy, combination of Methyl {5-[2-chloro-2-methylphenyl)-1-hydroxy-3-oxo-2,3-dihydro-1H-isoindol-1-yl]-1H-benzimidazol-2-yl}carbamate and CTLA-4 mAb resulted in 4 out of 8 complete regressions. These results are similar to those observed in the CT-26 colon carcinoma model (Methyl {5-[2-chloro-2-methylphenyl)-1-hydroxy-3-oxo-2,3-dihydro-1H-isoindol-1-yl]-1H-benzimidazol-2-yl}carbamate=1 CR; CTLA-4 mAb+2CR, Combination+5 CRs).

In summary, synergistic effects were observed with the concurrent treatment of CTLA-4 mAb+BRAF inhibitor in a murine SA1N fibrosarcoma as shown in FIG. 4.

### Example 4

### Method of Confirming the Effect of the Combination of a BRAF Inhibitor with a Co-Stimulatory Pathway Modulator on Tumor Growth in a Murine Antigen-Induced T-Cell Proliferation Model

Previously, the unexpected expansion of antigen-specific T cells with the use of Methyl {5-[2-chloro-2-methylphenyl)-1-hydroxy-3-oxo-2,3-dihydro-1H-isoindol-1-yl]-1H-benzimidazol-2-yl}carbamate+CTLA-4 mAb in an OVA adoptive transfer model was observed (see Example 2). Thus, a second study was designed to confirm these initial observations to determine the dose effect of Methyl {5-[2-chloro-2-methylphenyl)-1-hydroxy-3-oxo-2,3-dihydro-1H-isoindol-1-yl]-1H-benzimidazol-2-yl}carbamate and to determine whether this phenomenon could also be elicited by other RAF inhibitors. In addition, EVRI, an EGFR/VEGFR inhibitor which inhibits the MAPK pathway was also included in this experiment.

Results from the second study in the OVA model showed that the following a) CTLA-4 mAb expanded OVA-specific T-cell proliferation; b) addition of Methyl {5-[2-chloro-2-methylphenyl)-1-hydroxy-3-oxo-2,3-dihydro-1H-isoindol-1-yl]-1H-benzimidazol-2-yl}carbamate to CTLA-4 mAb enhanced CTLA-4 blockade OVA-specific T cell expansion in a dose-related manner; c) the RAF inhibitor EXT-000153 also enhanced the response elicited by CTLA-4 mAb; and d) EVRI did not inhibit or enhance the CTLA-4 mAb effect (similar to prior observations with erlotinib—data not shown).

In summary, these results suggest that enhanced expansion of antigen-specific T cells by the combination of Methyl {5-[2-chloro-2-methylphenyl)-1-hydroxy-3-oxo-2,3-dihydro-1H-isoindol-1-yl]-1H-benzimidazol-2-yl}carbamate or EXT000153+CTLA-4 mAb may be the result of BRAF inhibition. In the past two months, publications from three different laboratories showed that while BRAF inhibitors are very effective in suppressing BRAF mutant-driven tumor cell proliferation and growth in preclinical and clinical studies, the same compounds appeared to induce MEK/ERK pathway activation in cells with wild type RAF (see Heidorn et al., Poulikakos et al; and Hatzivassiliou et al.). Mechanistically, it was suggested that BRAF inhibitors activate CRAF through the formation of B-RAF/C-RAF heterodimer complexes resulting in activation of the RAF-MEK-ERK pathway. Results from these studies provide a plausible explanation for our observations on the potentiation of antigen-specific T-cell expansion by Methyl {5-[2-chloro-2-methylphenyl)-1-hydroxy-3-oxo-2,3-dihydro-1H-isoindol-1-yl]-1H-benzimidazol-2-yl}carbamate and EXT-000153 in vivo. While results from the studies presented support the use of BRAF inhibitors in combination with ipilimumab for the treatment of patients with BRAF-mutant melanoma, additional studies will be conducted to dissect the biochemical mechanism triggered in activated/proliferating T cells.

The present invention is not limited to the embodiments specifically described above, but is capable of variation and modification without departure from the scope of the appended Claims.

It will be clear that the invention may be practiced otherwise than as particularly described in the foregoing description and examples. Numerous modifications and variations of the present invention are possible in light of the above teachings and, therefore, are within the scope of the appended Claims.

The entire disclosure of each document cited (including patents, patent applications, journal articles, abstracts, laboratory manuals, books, GENBANK®Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

What is claimed is:

1. A method for the treatment of proliferative diseases, including cancer, which comprises administration to a mammal in need thereof a synergistically, therapeutically effective amount of an anti-CTLA-4 agent with methyl {5-[2-chloro-2-methylphenyl)-1-hydroxy-3-oxo-2,3-dihydro-1H-isoindol-1-yl]-1H-benzimidazol-2-yl}carbamate.

2. The method according to claim 1, wherein said administration comprises the sequential administration of (i) one or more cycles of methyl {5-[2-chloro-2-methylphenyl)-1-hydroxy-3-oxo-2,3-dihydro-1H-isoindol-1-yl]-1H-benzimidazol-2-yl}carbamate; followed by the administration of (ii) one or more cycles of said anti-CTLA-4 agent or a combination comprising said methyl {5-[2-chloro-2-methylphenyl)-1-hydroxy-3-oxo-2,3-dihydro-1H-isoindol-1-yl]-1H-benzimidazol-2-yl}carbamate and said anti-CTLA-4 agent.

3. The method according to claim 1 or claim 2 wherein the anti-CTLA-4 agent(s) is selected from the group consisting of ipilimumab and tremelimumab.

4. The method according to claim 1 or claim 2, wherein said method is for the treatment of cancerous solid tumors.

5. The method according to claim 1 or claim 2, wherein said method is for the treatment of refractory tumors.

6. The method according to claim 1 or claim 2, wherein the anti-CTLA-4 agent is selected from the group consisting of an anti-CTLA-4 antibody, an anti-CTLA-4 adnectin, an anti-CTLA-4 RNAi, single chain anti-CTLA-4 antibody fragments, domain anti-CTLA-4 antibody fragments, and an anti-CTLA-4 antisense molecule.

7. A method for the treatment of proliferative diseases, including cancer, which comprises administration to a mammal in need thereof a synergistically, therapeutically effective amount of an anti-CTLA-4 agent with PLX-4032.

8. The method according to claim 7, wherein said administration comprises the sequential administration of (i) one or more cycles of a BRAF inhibitor; followed by the administration of (ii) one or more cycles of said anti-CTLA-4 agent or a combination comprising said BRAF inhibitor and said anti-CTLA-4 agent.

9. The method according to claim 7 or claim 8 wherein the anti-CTLA-4 agent(s) is selected from the group consisting of ipilimumab and tremelimumab.

10. The method according to claim 7 or claim 8, wherein said method is for the treatment of cancerous solid tumors.

11. The method according to claim 7 or claim 8, wherein said method is for the treatment of refractory tumors.

12. The method according to claim 7 or claim 8, wherein the anti-CTLA-4 agent is selected from the group consisting of an anti-CTLA-4 antibody, an anti-CTLA-4 adnectin, an anti-CTLA-4 RNAi, single chain anti-CTLA-4 antibody fragments, domain anti-CTLA-4 antibody fragments, and an anti-CTLA-4 antisense molecule.

13. The method according to claim 7 or claim 8 wherein the BRAF inhibitor is selected from the group consisting of methyl {5-[2-chloro-2-methylphenyl)-1-hydroxy-3-oxo-2,3-dihydro-1H-isoindol-1-yl]-1H-benzimidazol-2-yl}carbamate; a V600E BRAF inhibitor, and PLX-4032.


SEQUENCE LISTING (4 sequences)

SEQ ID NO: 1 - protein, 108 aa, homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 2 - protein, 118 aa, homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 3 - protein, 9 aa, homo sapiens
IMDQVPFSV

SEQ ID NO: 4 - protein, 9 aa, homo sapiens
YLEPGPVTV